(19) United States
(12) Patent Application Publication
(10) Pub. No.: US 2020/0297649 A1
(43) Pub. Date: Sep. 24, 2020

(54) DEVICES AND METHODS FOR DELIVERY OF OXYGEN TO A WOUND

(71) Applicant: RESPIROGEN, INC., Boulder, CO (US)

(72) Inventors: Paul MOUNTFORD, Boulder, CO (US); Mark BORDEN, Boulder, CO (US); Robert T. Scribner, Boulder, CO (US); Robert M. Scribner, Boulder, CO (US)

(21) Appl. No.: 16/900,659

(22) Filed: Jun. 12, 2020

Related U.S. Application Data
(63) Continuation of application No. PCT/US18/64947, filed on Dec. 11, 2018.
(60) Provisional application No. 62/597,259, filed on Dec. 11, 2017.

(51) Int. Cl. A61K 9/50; A61K 9/70; A61K 33/00; A61K 9/00

(52) U.S. Cl. CPC A61K 9/5015; A61K 9/0014; A61K 33/00; A61K 9/7084

(57) ABSTRACT

Methods for treating cutaneous conditions and dermatoses such as disorders of the skin, subcutaneous tissues, mucous membranes, poorly vascularized tissues and/or other tissue disorders, including erosions, fissures, transient and/or chronic sores, burns, wounds, ulcers, lesions and infections. In particular embodiments, treatments include methods for improving skin and related tissue healing and repair using oxygen microbubbles (OMBs) and/or various formulations and/or compounds incorporating OMBs in combination with various other medicaments and/or tissue implants.

### CROSS REFERENCE TO RELATED APPLICATIONS

This application is a continuation application of PCT Application Serial No. PCT/US18/64947 entitled “Devices and Methods for Delivery of Oxygen to a Wound,” filed Dec. 11, 2018, which claims priority to U.S. Provisional Patent Application No. 62/597,259 entitled “Devices and Methods for Delivery of Oxygen to a Wound,” filed Dec. 11, 2017, the disclosures of which are each incorporated by reference herein in their entireties.

### FIELD OF THE INVENTION

The various embodiments herein pertain to the field of treating cutaneous conditions and dermatoses such as disorders of the skin, subcutaneous tissues, mucous membranes and/or other tissue disorders, including improvements to skin and related tissue healing and repair of various conditions including erosions, fissures, transient and/or chronic sores, wounds, ulcers, lesions and/or infections.

### BACKGROUND OF THE INVENTION

### Description of the Related Art

A cutaneous condition is a medical condition that affects the integumentary system, which is the organ system that encloses the body and includes skin, hair, nails, and related muscle and glands. The skin of an adult weighs an average of between 4 to 5 kilograms (8.8 to 11 pounds), covers an area of approximately 22 square feet, and includes three distinct layers: the epidermis, dermis, and subcutaneous tissue. There are two main types of human skin: (1) glabrous skin, which is the non-hairy skin on the palms and soles (i.e., palmoplantar surfaces), and (2) hair-bearing skin, which incorporates hairs in structures called pilosebaceous units, each with hair follicles, sebaceous glands, and associated arrector pili muscles.

The epidermis is the most superficial layer of skin and is a squamous epithelium with several strata: the stratum corneum, stratum lucidum, stratum granulosum, stratum spinosum, and stratum basale. Nourishment to the various layers is provided via diffusion from the dermis, as the epidermis is without a direct blood supply. The epidermis contains four cell types: keratinocytes, melanocytes, Langerhans cells, and Merkel cells. Keratinocytes are the major component of the epidermis, constituting roughly 95 percent of the cells therein. The stratified squamous epithelium is maintained by cell division within the stratum basale, in which differentiating cells slowly displace outwards through the stratum spinosum to the stratum corneum, where cells are continually shed from the surface. The stratum basale is a single layer of cells, closest to the dermis. It is usually only in this layer that cells divide. Some of the dividing cells move up to the next layer.

The prickle cell layer (stratum spinosum) is the next layer (8-10 layers of cells). The cells in these layers have lots of desmosomes, which anchor the cells to each other, and contain thick tufts of intermediate filaments (keratin). When the cell shrinks slightly, such as during fixation, the desmosomes from neighboring cells remain tightly bound to each other, and these connections look like ‘prickles’ or ‘spines’, hence the name prickle cells.

The granule cell layer (stratum granulosum) is the next layer (3-5 layers of cells). As the cells move up into this layer, they start to lose their nuclei and cytoplasmic organelles, and turn into the keratinised squames of the next layer. The granules contain a lipid rich secretion, which acts as a water sealant.

In thick skin, a fifth layer (stratum lucidum) is sometimes identified—between the stratum granulosum and stratum corneum layer. It is a thin transparent layer, difficult to recognize in routine histological sections.

The keratinised squames layer (stratum corneum) is the final layer. These are layers of dead cells, reduced to flattened scales, or squames, filled with densely packed keratin. In histological sections these cells are flat and hard to see. The squames on the surface of this layer flake off on a regular basis (making up the main content of household dust).

In normal skin, the rate of production generally equals the rate of loss—i.e., it normally takes about two weeks for a cell to migrate from the basal cell layer to the top of the granular cell layer, and an additional two weeks to cross the stratum corneum. This continuous replacement of cells in the epidermal layer of skin is important. The epidermal layer of the skin and the digestive tract are the two tissues that are directly exposed to the outside world, and therefore are most vulnerable to its damaging effects. In both, there is constant proliferation of cells in the bottom layer (stratum basale) which constantly move up to the top where they are lost. This means damaged cells are continually shed and replaced with new cells.

The dermis is the layer of skin between the epidermis and subcutaneous tissue, and includes two sections, the papillary dermis and the reticular dermis. The superficial papillary dermis interdigitates with the overlying rete ridges of the epidermis, between which the two layers interact through the basement membrane zone. Structural components of the dermis include collagen, elastic fibers, and extrafibrillar matrix (otherwise referred to as “ground substance”). Within these components are the pilosebaceous units, arrector pili muscles, and the eccrine and apocrine glands. The dermis normally contains two vascular networks that run parallel to the skin surface (i.e., one superficial and one deep plexus), which are connected by vertical communicating vessels. The function of blood vessels within the dermis is at a minimum fourfold: to supply nutrition, to regulate temperature, to modulate inflammation, and to participate in wound healing.

The subcutaneous tissue or “hypodermis” is a layer of fat between the dermis and underlying fascia, and this tissue can be further divided into two components, the actual fatty layer (i.e., panniculus adiposus) and a deeper vestigial layer of muscle (i.e., panniculus carnosus). The main cellular component of this tissue is the adipocyte, or fat cell. The structure of this tissue is composed of septal (i.e. linear strands) and lobular compartments, which differ in microscopic appearance. Functionally, the subcutaneous fat insulates the body, absorbs trauma, and serves as a reserve energy source.

Aside from wounds, fissures, lesions and/or infections of the skin, one particular class of cutaneous conditions that affects a substantial portion of the general population are skin ulcers. An ulcer is a sore on the skin or mucous membrane of a patient, generally accompanied by the disintegration of tissues. Ulcers can result in the complete loss of the epidermis, and often portions of the dermis and even subcutaneous fat. Ulcers are most common on the skin of the lower extremities and in the gastrointestinal tract. An ulcer that appears on the skin is often visible as an inflamed tissue with an area of reddened skin.

Ischemic skin ulcers and other wound types can occur when there is poor blood flow in and/or adjacent to a region of skin. Poor blood flow can cause various skin cells to die and damage other tissues. Ulcers can also be caused by exposure to heat or cold and/or irritation, which can cause a sore to form. Ulcers can also be caused due to a lack of mobility, which can cause prolonged pressure on the tissues. This stress in the blood circulation is transformed to a skin ulcer, commonly known as bedsores or decubitus ulcers. Skin ulcers can appear as open craters, often formed in a round shape, with layers of skin that have eroded. The skin around the ulcer may be red, swollen, and tender. Patients may feel pain on the skin around the ulcer, and fluid may ooze from the ulcer. In many cases, ulcers can become infected, which can include the formation of pus. In some cases, ulcers can bleed and patients can experience fever.

Ulcers typically develop in stages. In stage 1 the skin is red with soft underlying tissue. In the second stage the redness of the skin becomes more pronounced, swelling appears, and there may be some blisters and loss of outer skin layers. During the next stage, the skin may become necrotic down through the deep layers of skin, and the fat beneath the skin may become exposed and visible. In stage 4, deeper necrosis usually occurs, the fat underneath the skin is completely exposed, and the muscle may also become exposed. In the last two stages the sore may cause a deeper loss of fat and necrosis of the muscle; in severe cases it can extend down to bone level, destruction of the bone may begin, and there may be sepsis of joints and an ultimate need for amputation of the affected limb.

Ulcers of the lower legs represent a serious challenge for medicine, especially in the case of diabetic patients. Ulcers of the lower legs are formed mainly as a consequence of chronic venous insufficiency and/or in diabetic patients (i.e., diabetic foot/leg ulcers) as a complication of decreased vasculature and/or microvasculature and a peripheral neuropathy that permits increased trauma to pass unnoticed because of decreased sensation (i.e., diabetic angiopathy, macroangiopathy, microangiopathy and/or neuropathy). Healing of the various types of ulcers is often difficult because insufficient or absent circulation blocks transport of oxygen and nutrients to the cells. As a result, undernourished cells die and necrosis of tissue develops. The lack of circulation also blocks the removal of cell debris and further impedes normal healing processes. Without a healthy, intact skin barrier, the surface of the ulcer is open for infections, which add to the treatment problems. Moreover, ulcers are different from other wounds because whereas normal wounds heal spontaneously over a certain period of time, ulcers, once started, tend to increase in size and wound depth instead of healing. The defective circulation associated with ulcers can cause malnutrition and finally necrosis of the tissue. This in turn, causes a progression of the ulceration which often cannot be compensated by the normal processes of skin repair.

Even when ulcers heal, they often heal very slowly, and in many cases seem not to heal at all. In general, ulcers that heal within 12 weeks are classified as acute, and longer-lasting ones as chronic. Chronic ulcers can be painful, and most patients complain of constant pain at night and during the day. Chronic ulcer symptoms usually include increasing pain, friable granulation tissue, foul odors, and wound breakdown instead of healing.

Treatment of ulcers generally revolves around a desire to promote the normal healing process while avoiding infection of the ulcer, as symptoms tend to worsen dramatically once the wound has become infected. A vast selection of topical formulations is directed to treatment of ulcers, which in most cases are combinations of bacteriostatic or bactericidal drugs, vitamins, herbal constituents, absorbing powders, proteolytic enzymes and others. Treatment typically includes various steps to remove any excess discharge, maintain a moist wound environment, control the edema, and ease pain caused by nerve and tissue damage. The wound or ulcer is usually kept clear of dead tissue through surgical debridement and, in some cases, the creation of skin flaps and/or skin grafting may become necessary. Each treatment method can significantly affect the progression and rate of healing as well as the type of tissues formed. In the case of lower extremity ulcers, special exercises and/or compression bandages may be recommended to stimulate circulation of blood in the lower legs. In addition, it is often desirous to offload the treated extremity to prevent further tissue damages and/or promote healing of the damaged tissues.

In many cases, an underlying cause of the ulcer, and/or a major factor contributing to its inability to heal in a timely manner, is impaired blood circulation and/or poor blood flow in and/or adjacent to the region of skin containing the ulcer, typically leading to poor oxygenation of the damaged skin region and/or underlying anatomical structures. Although skin ulcers do not seem of great concern at a first glance, they are worrying conditions, especially in people suffering from diabetes, as they are at risk of developing diabetic neuropathy. Moreover, it is likely that a person who has had a skin ulcer will eventually have it again.

### SUMMARY OF THE INVENTION

Various aspects of the present invention include the realization of a need for improved diagnosis and/or treatment of ulcers and skin wounds, especially skin ulcers, burns (i.e., due to excessive heat, cold, chemical, radiation, wind and/or otherwise induced) as well as other wounds resulting from and/or experiencing delayed healing due to ischemic conditions. In various embodiments, wounds and injuries, including skin ulcers and/or other types of damaged skin surfaces, can be treated by application of a topical compound which includes oxygenated microbubble formulations, including microbubble formulations that incorporate lipid formulations, which may be applied using a variety of delivery modalities and/or treatment algorithms to desirably promote shorter time to healing.

In general, wounds heal more quickly and are often less complicated by infection when in a moist environment. A wound's exudate is rich in cytokines, platelets, white blood cells, growth factors, matrix metalloproteinases (MMPs), and other enzymes. Most of these factors promote healing via fibroblast and keratinocyte proliferation and angiogenesis, while others, such as leukocytes and toxins produced by bacteria, inhibit the healing process. Moreover, it has been reported that local concentrations of growth factors [platelet-derived growth factor beta (PDGF-beta), transforming growth factor beta] are low in patients with chronic wounds. In many embodiments, an ideal dressing should be free from contaminants, be able to remove excess exudates and toxic components, maintain a moist environment at the wound-dressing interface, be impermeable to microorganisms, allow gaseous exchange, and, finally, should be easily removed and cost-effective. Various dressings are available that are intended to prevent infection and enhance wound healing, and several studies support their effectiveness for this purpose.

In various embodiments, the invention can include lipid encapsulated oxygen microbubbles (OMBs) which are delivered topically or by continuous flow and delivery to and across the surface of a wound. In some embodiments, the delivery may take place by applying the OMBs to the surface of a dressing which is applied to the wound directly. In other embodiments, the dressing may be provided with inflow and outflow points at opposing locations on or adjacent to the wound. Under positive pressure, OMBs move from the inlet to the outlet across the wound, providing an enhanced oxygen level for interaction with the tissue of the wound. Flow of the OMBs may be continuous, intermittent, or varied according to desired treatment parameters. In still another embodiment, the OMBs may be combined with medications beneficial to antibacterial treatment, stem cell formulations, growth factors, or additional types of medicaments for the promotion of wound healing and/or for reduction in pain and/or inflammation.

On various embodiments, the OMB formulations may comprise a dried powder, a gel, an ointment, a lotion, a cream, an oily solution, a suspension, or a semi-solid, and may be applied directly to the surface of the wound and/or impregnated or carried by a dressing, bandage and/or other medical treatment applied to the wound. A dosage of the composition may be administered continuously over a period of multiple days, periodically over an interval of multiple days, may be administered once a day or may be administered multiple times a day, or in the case of a bandage or dressing containing a reservoir of treatment material, may comprise an essentially continuous or periodic “re-application” over a period of time. The number of administrations per day may be, for example, 2, 3, 4, 5, 6 or more. That is, the administration can be applied on a periodic basis, which could include application each day over the course of a treatment period. The treatment period may extend over a period of time necessary to heal one or more wounds or ulcers, which may include treatment durations of 14, 28, 42, 70, 91, or 140 or more days.

The topical application of an OMB formulation to the surface of a wound and/or the surrounding epidermal skin surface will desirably induce improved healing reactions in one or more of the tissue layers underlying and/or adjacent to the damaged portion(s) of the epidermis, which can potentially increase localized oxygen availability for cells proximate to the OMB by intercellular or transappendageal pathways, and may even facilitate oxygenation of tissues remote from the OMB formulation via blood flow transfer via the vascular network adjacent to the affected area. In various embodiments, the OMB may induce mitosis (i.e., cell division) or other healing responses of dermal fibroblasts, vascular endothelial cells and/or epidermal keratinocytes. Desirably, the OMB and/or related medicaments will enhance closure of the wound surfaces (i.e., from the wound margins and/or subsurface tissues) while concurrently improving the condition of the underlying tissues and/or vascular network supporting the surrounding layers of the skin and underlying anatomical structures.

In various embodiments, such as where skin or other tissue grafts may be anticipated, the topical application of the OMB formulation will desirably provide supplemental oxygenation and/or initiate an angiogenic cascade in one or more of the tissue layers underlying and/or adjacent to the wound, thereby preparing the wound bed and/or surrounding tissue margins for receiving the potential graft material. When the graft material is placed adjacent to and/or in contact with the wound bed during the graft implantation procedure, the wound bed and/or adjacent tissues will desirably be capable of readily providing nutrients (i.e., via diffusion) to keep the skin graft alive, while concurrently allowing blood vessels to begin to grow from the wound bed into the graft. By the time the graft may no longer be able to survive by diffusion of nutrients alone (which can occur as soon as within a few days after graft implantation), with the OMB and/or any newly formed vascular network desirably providing supplemental oxygenation and/or nutrition, with the vasculature (and attendant diffusion therefrom and/or thereto) eventually becoming the primary mechanism for providing oxygen and nutrients to the graft. If desired, the graft material may be “loaded” with OMB containing substances in a similar manner, either prior to, concurrent with and/or after implantation in the wound bed.

In various other embodiments, the topical application of a formulation containing OMBs to the surface/subsurface of a skin wound and/or surrounding healthy tissues has the potential for “slowing down” and/or halting the process of ulceration for a patient, which might potentially include localized and/or systemic effects that may alleviate various symptoms of the underlying diseases in a systemic manner—including the effects of prolonged compression (i.e., by being bedridden), chronic venous insufficiency and/or diabetes—by reducing, preventing and/or reversing further deterioration of circulation inside the lower legs. Even when a progression of damage may only be slowed and/or temporarily affected by the treatment, such treatment has the potential for slowing the irreversible degradation of the blood vessels and/or skin and related tissues, with attendant effects on the healing process.

Some embodiments can include the various treatments described herein in combination with various prosthesis designs to maintain the OMBs in a desired location and/or position, and/or protect the damaged skin during some or all of the course of treatment. In various embodiments involving lower extremity skin ulcers, protective bandages can be utilized to desirably protect and/or “shield” the damaged tissue while concurrently applying a therapeutic compound to the surface of the damaged tissue.

In various additional embodiments, methods of assessing and treating damage, wounds and/or ulcers to the skin can include the steps of assessing the damaged tissue and related underlying anatomical areas, assessing the relevant tissue regions, developing a treatment plan and protecting and/or treating the damaged tissue region.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention:

FIG. 1 depicts a cross-sectional view of epidermal tissues;

FIG. 2 depicts one exemplary embodiment of a bandage system for use with various OMB compounds;

FIG. 3 depicts another alternative embodiment of a bandage system for use with various OMB compounds;

FIG. 4 depicts another alternative embodiment of a bandage system for use with various OMB compounds;

FIGS. 5A through 5C depict various views of one embodiment of an insert or pad that can serve as a “reservoir” of an OMB formulation and/or compound;

FIG. 5D depicts a storage device or “peel pouch” for containing the insert of FIGS. 5A through 5C;

FIG. 6A depicts an alternative embodiment of a prosthesis for use in treating skin ulcers and other wounds with an OMB formulation and/or related medicaments; and

FIG. 6B depicts the compression-type prosthesis of FIG. 6A positioned about a patient's lower extremity.

### DETAILED DESCRIPTION OF THE INVENTION

The following description is presented to enable any person skilled in the art to make and use the invention. Various modifications to the embodiments described will be readily apparent to those skilled in the art, and the generic principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclose herein. To the extent necessary to achieve a complete understanding of the invention disclosed, the specification and drawings of all issued patents, patent publications, and patent applications cited in this application are incorporated herein by reference. Although some embodiments are described below, these are merely representative and one of skill in the art will be able to extrapolate numerous other applications and derivations that are still within the scope of the invention disclosed.

It has been determined that Oxygen MicroBubbles (OMBs) and formulations containing OMBs possess a remarkable ability to oxygenate and/or moisturize tissues in and/or proximate to the integumentary system to promote and/or heal damage thereto. The integumentary system is the organ system that encloses the body and includes skin, hair, nails, and related muscle and glands. Because much of the integumentary system relies upon diffusive transport of oxygen and nutrition (and also for waste removal) from the vascular system in the body, even minor degradation of the tissues and/or the vascular system in the localized region supporting such diffusive transport can severely reduce the integumentary system's ability to protect the body from various kinds of damage, such as acting as a barrier to the external environment, protecting against loss of water, cushioning and protecting deeper tissues, excreting wastes, and/or regulating temperature. Where a significant interruption to the tissues and/or underlying vascular system occurs, the consequences for the overlying integumentary system (and concurrently the overall health of the organism) can be catastrophic, as a damaged or degraded integumentary system poses a significant risk to the organism of disease, infection and ultimately death.

Because the outer surface of the skin is typically an avascular structure, much of the anatomy of the integumentary system relies upon diffusion for oxygen, nutrition, and waste removal. The oxygen and nutrients required to maintain cellular function and viability are supplied to the skin surface by capillary vessels and microvasculature in the subsurface tissue layers proximate to the surface layers. In addition, waste products can be removed via similar mechanisms.

Specifically, while the deeper dermal layers of skin contain heavily vascularized channels, the shallower and/or surface layers of the epidermis rely mainly upon diffusive flow from the deeper layers to transport oxygen and nutrients from the blood to the cells of these layers, as well as the transport of various waste products from the cells back to the blood for removal and/or reuse by various other organs. The oxygen, glucose and other nutrients are “dropped off” from the capillaries, and then the nutrients “diffuse” (or otherwise move through the adjacent tissues without being transported in blood vessels, including movement by intercellular and transappendageal pathways) to the adjacent skin cells.

Once glucose and oxygen leave the capillaries, passive diffusion becomes the mechanism of nutrient transport through the intervening anatomical layers. A large concentration gradient may be required for optimal diffusion. The concentration gradient is determined by the utilization of the nutrients by the surrounding tissue population and the concentration of nutrients delivered to the localized anatomical region by the microcirculation. Thus, any decrease in the population of the microvasculature has the potential to create metabolic derangement within the skin layers, leading to degeneration.

Once the oxygen and nutrients reach the cell, they are taken up and utilized for the manufacture of materials that make up the skin layers. If the cells do not receive enough oxygen, the manufacturing process typically stops and/or significantly reduces. Similarly, as the nutrient supply is cut off, the cells may begin to die, and the thickness and integrity of the skin tissue can begin to breakdown, which may predispose the skin to degeneration and/or damage.

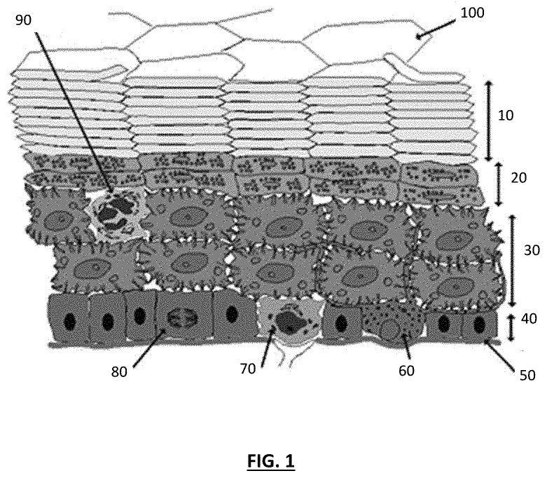

Oxygen transport from the vasculature to a cell in the tissue is a two-step process. First, materials flow near to their destination via blood vessels. Then they cover the remaining distance from the blood vessels to the cells primarily via diffusion. The time required for diffusion over large distances is often much longer than that needed for perfusive flow, because diffusion times grow as the square of distance whereas flow times are merely proportional to distance. Under normal conditions, blood is distributed to the capillary bed through an orderly tree-like system of conduits. From there, normal diffusion distances are highly regulated, often to distances less than 50 or 100 μm, and it is generally accepted that the distance that oxygen and other nutrients can diffuse into a given tissue before being metabolized by surrounding cells establishes a maximum distance for “healthy” cells to exist (i.e., “unstressed” cells receiving a desired level of nutrients and oxygen). For example, in the shallower layers of the integumentary system, the epidermal cells with the highest metabolic demand are found closest to the basal lamina, where the diffusion distance is typically shortest, while the surface or “superficial cells” which are more remotely located from the vasculature typically are less active and/or are generally inert or dead (see FIG. 1). In this drawing are included keratinized squames 10, a granule cell layer 20, prickle cell layers 30, a basal cell layer 40, a basil lamina 50, a melanocyte 60, a Merkel cell 70, a dividing cell 80, a Langerhan's cell 90 and a squame about to flake off of the skin surface 100.

Glucose and oxygen are extremely important to the function and viability of the skin cells. Regardless of the complex interactions taking place in the various skin layers, however, the fact remains that the supply of nutrients, the removal of waste and the overall health of the integumentary system normally require an intact vascular supply and microvascular capillary network.

Skin is the largest and the most frequently traumatized organ system in the body. Skin injuries are one of the chief causes of death in North America for people between the ages of 1 and 44. In much of the population, the normally healthy vasculature within the dermal layers underlying the integumentary system may be compromised to some degree for a variety of reasons (which can include simple age-related degradation of the patient's body), but for many individuals the level of compromise is of little or no clinical consequence. However, for other individuals, the level of vascular compromise (i.e., systemic or localized) can significantly affect the health and well-being of the patient.

For example, over 5% of individuals over the age of 50 suffer from a vascular deficiency condition known as Peripheral Artery Disease (PAD), in which one or more arteries of the extremities becomes clogged with plaque. PAD most commonly occurs when extra cholesterol and/or other fats circulating in the blood collect in the walls of the arteries that supply blood to the limbs. This buildup, called atherosclerosis and consisting of plaque, narrows the arteries, often reducing or blocking the flow of blood, which can occur in a localized region, can affect an entire extremity, or in extreme cases can result in systemic consequences. In fact, the number of individuals suffering from PAS is likely a much higher percentage than 5%—while a diagnosis of PAD generally identifies that the degenerative vascular condition has reached a significantly advanced condition impacting quality of life, many patients not yet fully diagnosed with PAD will already be suffering from concomitant occlusions and/or blockages in the vasculature and/or microvasculature of one or more extremities as “part and parcel” of the normal disease progression.

Lower Extremity Arterial Disease (LEAD) is a subclass of PAD that is clinically identified by intermittent claudication and/or absence of peripheral pulses in the lower legs and feet. These clinical manifestations reflect decreased arterial perfusion of the extremity. The incidence and prevalence of LEAD increase with age in both diabetic and non-diabetic subjects and, in those with diabetes, increase with duration of diabetes. A common complaint of patients suffering from LEAD, and especially true of diabetic patients, is the patient's proneness to infection, ulcerations and poor healing of skin sores and ulcers. Moreover, LEAD in diabetes is compounded by the presence of peripheral neuropathy and insensitivity of the feet and lower extremities to pain and trauma. The combination of impaired circulation and impaired sensation in such patients can easily lead to ulceration and infection, often progressing to osteomyelitis and gangrene which may necessitate amputation of part or all of the affected extremity.

In the case of diabetes, the disease burden of the diabetic foot that develops an ulcer is substantial. From the estimated 24 million Americans who have diabetes, the annual prevalence of foot ulcers in this population ranges from 4-10%, or approximately 1 million to 2.5 million subjects suffering with foot ulcers each year. Diabetic foot is one of the most significant and devastating complications of diabetes, and is defined as a foot affected by ulceration that is associated with neuropathy and/or peripheral arterial disease of the lower limb in a patient with diabetes. The prevalence of diabetic foot ulceration in the diabetic population is 4-10%; the condition is more frequent in older patients. It is estimated that about 5% of all patients with diabetes present with a history of foot ulceration, while the lifetime risk of diabetic patients developing this complication is 15%. The majority (60-80%) of foot ulcers will heal, while 10-15% of them will remain active, and 5-24% of them will finally lead to limb amputation within a period of 6-18 months after the first evaluation. Neuropathic wounds are more likely to heal over a period of 20 weeks, while neuroischemic ulcers take longer and will more often lead to limb amputation. It has been found that 40-70% of all nontraumatic amputations of the lower limbs occur in patients with diabetes. Furthermore, many studies have reported that foot ulcers precede approximately 85% of all amputations performed in diabetic patients. Complications from non-healing ulcers, including infection and gangrene, are the leading causes of hospitalization in patients with diabetes mellitus. The most costly and feared consequence of a foot ulcer is amputation of the limb. Each year, an estimated 82,000 limb amputations are performed on diabetic patients in the U.S. The risk of foot ulceration and limb amputation increases with age and the duration of diabetes.

The effects of LEAD and diabetes together account for approximately 50% of all nontraumatic amputations in the United States, and it is acknowledged that a secondary amputation within several years after the first is exceedingly common. Moreover, mortality is increased in patients with LEAD, particularly if foot ulcerations, infection, or gangrene occur, and three-year survival after an amputation is <50%. Prevention is an important component of LEAD management, because by the time LEAD becomes clinically manifest, it may be too late to salvage an extremity, or it may require more costly resources to improve the circulatory health of the extremity. While surgically invasive revascularization procedures of the larger arteries can improve perfusion and flow to the lower extremity, such procedures are often not recommended in a large proportion of patients, and even where successful have not had an appreciable reduction in the frequency of amputation experienced by revascularized patients.

The prevention of diabetic foot is crucial, considering the negative impact on a patient's quality of life and the associated economic burden on the healthcare system. The use of OMBs and formulations containing OMBs to promote shorter healing times for wound treatment, and effect healing in chronic non-healing wounds, may be useful to primary care physicians, nurses, podiatrists, diabetologists, and vascular surgeons, as well as all healthcare providers involved in the prevention or management of diabetic foot ulcers, and the treatment of other wound pathology.

Another common disorder of the integumentary system are pressure sores or ulcers, commonly referred to as “bedsores.” One of the most prevalent skin injuries affecting a large percentage of the patient population, bedsores are injuries to the skin and underlying tissue resulting from prolonged pressure on the skin, which are caused by pressure against the skin that limits blood flow to the skin and nearby tissues. In these cases, a localized area of tissue necrosis develops when the soft tissue is compressed for a prolonged period (often between a bony prominence and an external surface), forming the bedsore. Bedsores can range from superficial inflammation that extends into the dermis to an extensive ulcer occasionally involving underlying bone.

Bedsores are especially prevalent in areas of the body that aren't well-padded with muscle or fat and that lie over a bone, such as the spine, tailbone, shoulder blades, hips, heels and elbows. When the skin and underlying tissues are trapped between the underlying bone and a surface that presses on the skin (such as a wheelchair or a bed surface), this pressure may be greater than the pressure of the blood flowing in the capillaries and/or other vessels that deliver oxygen and other nutrients to the tissues, potentially impeding and/or halting the flow of such materials. When the pressure is sustained for a sufficient period of time, which can be as little as a few hours, the skin and underlying structures can become damaged and/or eventually die. Other factors contributing to the severity of bedsores can include friction damage, if the skin is being dragged across a surface during movement, and shear damage (i.e., compression, tension and/or shear forces) applied to the skin and underlying tissues—motion that may injure tissue and blood vessels, making the site more vulnerable to damage from sustained pressure.

Bedsores and other types of pressure ulcers are one of the most debilitating and costly problems associated with hospitalization, including surgical procedures involving long term care and rehabilitation, immobilization and/or disabling conditions such as spinal cord injury (SCI). Pressure ulcers can interfere with every aspect of a physically disabled individual's life, from active participation in the rehabilitation program to returning to an active role in the community. Pressure ulcers are found in 20-30% of individuals with SCI, 43% among nursing home residents, and 15% of persons with acute injuries, with people at risk of pressure ulcers and/or bedsores are those with a medical condition that limits their ability to change positions, requires them to use a wheelchair or confines them to a bed for a long time. In 2006, it was estimated that persons with SCI who have pressure ulcers incur hospital charges three to four times those of other individuals with SCI, and averaged at least an additional $48,000 in health care costs. In 2010 it was calculated that, for the most severe sores (i.e., grade 4), the average hospital treatment cost was more than $129,000 for hospital-acquired ulcers during one admission, and $124,000 for community-acquired ulcers over an average of 4 admissions sores (i.e., grade 4). Since hospital charges relate directly to the number of days in treatment, reducing the length of a hospital stay through more effective treatment of pressure ulcers, skin ulcers or other skin wounds could mean a significant savings for the patient, the health care delivery system, and the third party payer. Moreover, a nonsurgical treatment that promotes healing in a shorter time would reduce the hospital stay, recovery time, costs, and complications associated with surgical skin grafting. In addition, malpractice suits associated with the development of pressure ulcers average $250,000 per settlement, reportedly totaling at least $65,000,000 annual in the U.S. alone.

Regardless of the cause(s) of damage or wounds to the integumentary system, an important feature of a healthy integumentary system is the ability of the body to heal such damage or wounds. In normal, healthy patients, the epidermal skin layers typically exist in a “steady-state” equilibrium—forming a protective barrier against the external environment. An injury to the skin sets into motion a set of complex biochemical events in a closely orchestrated cascade, which seeks to repair the damage. The response to injury is an essential innate host immune response for restoration of tissue integrity. Tissue disruption in higher vertebrates desirably results in a rapid repair process leading to a fibrotic scar. Wound healing, whether initiated by trauma, microbes or foreign materials, proceeds via an overlapping pattern of events including (1) hemostasis and coagulation, (2) inflammation, (3) proliferation (including epithelialization and formation of granulation tissue), and (4) matrix and tissue remodeling. The process of repair is mediated in large part by interacting molecular signals, primarily cytokines, which motivate and orchestrate the manifold cellular activities which underscore inflammation and healing.

The specific cellular activities and interrelationships in the wound healing cascade are extremely complex, but as a relatively simplified explanation, the following steps occur. Within the first few minutes after a skin injury, platelets adhere to the site of injury, become activated, and aggregate (i.e., they join together); followed by activation of the coagulation cascade which forms a clot of aggregated platelets in a mesh of cross-linked fibrin protein. This clot stops active bleeding (i.e., “hemostasis”).

The initial injury also triggers an acute local inflammatory response followed by mesenchymal cell recruitment, proliferation and matrix synthesis. During the “inflammation” phase, bacteria and cell debris are phagocytosed and removed from the wound by white blood cells. Platelet-derived growth factors (stored in the alpha granules of the platelets) are released into the wound that cause the migration and division of cells during the proliferative phase. Failure to resolve such inflammation can lead to chronic non-healing wounds, whereas uncontrolled matrix accumulation, often involving aberrant cytokine pathways, can lead to excess scarring and fibrotic sequelae.

Clearance of debris, foreign agents and any infectious organisms promotes resolution of inflammation, apoptosis, and the ensuing repair response that encompasses overlapping events involved in granulation tissue, angiogenesis, and re-epithelialization. Within hours, epithelial cells begin to proliferate, migrate and cover the exposed area to restore the functional integrity of the tissue. Re-epithelialization is seen as critical to optimal wound healing, not only because of reformation of a cutaneous barrier, but also because of its role in wound contraction. This “proliferation” phase is characterized by angiogenesis, collagen deposition, granulation tissue formation, epithelialization, and wound contraction. In angiogenesis, vascular endothelial cells desirably form new blood vessels. In fibroplasia and granulation tissue formation, fibroblasts grow and form a new, provisional extracellular matrix (ECM) by excreting collagen and fibronectin. Concurrently, re-epithelialization of the epidermis occurs, in which epithelial cells proliferate and ‘crawl’ atop the wound bed, providing cover for the new tissue. Immature keratinocytes produce matrix metalloproteases (MMPs) and plasmin to dissociate from the basement membrane and facilitate their migration across the open wound bed in response to chemoattractants. The migration of epithelial cells occurs independently of proliferation, and depends upon a number of processes, including growth factors, loss of contact with adjacent cells, and guidance by active contact.

During wound contraction, myofibroblasts decrease the size of the wound by gripping the wound edges and contracting using a mechanism that resembles that in smooth muscle cells. When the cells' roles are close to complete, unneeded cells undergo apoptosis. During maturation and “remodeling,” collagen is remodeled and realigned along tension lines, and cells that are no longer needed are removed by apoptosis.

While the process of wound healing in the skin of a healthy individual is a relatively straightforward process, the same cannot be said for wound healing in the skin of an individual suffering the effects of oxygen deprivation and/or vascular compromise. The complex skin healing process in such compromised individuals is often very fragile, and is susceptible to interruption or failure at many points—leading to the formation of non-healing chronic wounds. There are a wide variety of factors that can interfere with skin healing and the formation of such non-healing chronic wounds, including diabetes, venous or arterial disease, infection, and metabolic deficiencies of old age. Faulty or impaired healing has been repeatedly labelled the most prominent factor in these lesions, and thus speeding up the rate of regenerative healing would be expected to reduce both the likelihood and effect of other secondary complications.

Of all the potential complications affecting the ability of the skin to heal, the condition of the underlying vascular support network is arguably one of the most important. Virtually every step in the wound healing process either relies upon and/or is directly influenced by the conditions of the underlying vasculature. In many cases, an underlying vascular abnormality and/or insufficiency can significantly reduce and/or eliminate the body's ability to heal a skin wound. For example, the vasculature is the cells' primary source of oxygen and nutrition, as well as a primary channel for waste removal. A lack of nutrition can inhibit or prevent normal repair and/or replacement of cellular structures, while insufficient oxygen can result in cell death. In a similar manner, a lack of sufficient waste removal can result in a buildup of wastes within and/or between the cells—potentially degrading and/or inhibiting the cells' ability to function and properly repair damage. Moreover, the vasculature is the primary transport pathway for numerous cells and materials necessary for protection of the organism and repair of the skin wound—so an interruption in the vascular transport mechanism means an interruption in the availability of these cells/materials as well.

Another factor that can significantly affect the ability of skin wounds to heal is the presence or absence of infection. Where vascular compromise is a concern, skin wounds can be predisposed to infection because of the underlying vasculopathy as well as a related immunopathy (i.e., diminished neutrophil function). Once an infection has become established in a skin wound, the underlying vascular and/or microvascular compromise can further complicate treatment, as phagocytic cells will have limited access to the region and systemic antibiotics will generally have a poor concentration in the infected tissues. Moreover, infected skin wounds heal much more slowly than their non-infected counterparts.

Currently, the most effective conservative methods of treating skin wounds, including small or large ulcers, involve removing pressure from the affected area, providing a dressing or other covering over the wound to collect wound exudate and protect and hydrate the wound area, and allowing the body to naturally heal the skin. However, it would be desirous to incorporate additional interventions that could facilitate these processes and possibly even speed up regenerative skin healing, so as to reduce the costs, length of medical treatment and morbidity commonly associated with pressure ulcers. An optimal intervention in many cases would desirably include an intervention that increases local availability of oxygen for various damaged tissues and which enhances and optimizes the ability of the skin to regenerate at a rate equal to or greater than normal reparative healing.

There is strong evidence that fibroblasts, endothelial cells, and keratinocytes are replicated at higher rates in an oxygen-rich environment. Moreover, leukocytes kill bacteria more effectively when supplied by oxygen. It is also known that fibroblasts from diabetic individuals show diminished cell turnover in comparison with those from nondiabetic persons. Based on these data, the present inventions desirably facilitate the administration of oxygen at higher concentrations to accelerate wound healing. This is a much simpler and cost-effect technique than hyperbaric oxygen therapy, which involves the intermittent administration of 100% oxygen at a pressure greater than atmospheric pressure at a specific location and/or at sea level. Unlike hyperbaric oxygen therapy, the present systems do not require 30 to 40 sessions in a sealed chamber with the patient contained within a high oxygen environment (which can include oxygen concentrations of 100% or concentrations approaching 100%, and in some other treatments can involve lower oxygen concentrations such as 24-40% oxygen—generally in excess of the normal atmospheric oxygen percentage of 21% oxygen) while the atmospheric pressure is increased to 2-3 atmospheres for a duration of 1-2 hours. Rather, the disclosed systems and methods of administering OMBs and related formulations to a wound site will allow for localized delivery of high concentrations of oxygen at specific wound sites for defined treatment protocols, with patients able to receive care in multiple points of delivery including in hospital, living centers, home, and ambulatory settings, enabling beneficial oxygen therapy without the need for chamber delivery and highly trained personnel or restriction to the clinical setting.

In various embodiments, the oxygen microbubble formulations as described herein can be comprise a stabilizing phospholipid shell and an oxygen gas core ranging from 21% or greater, to 22%, or greater to 23% or greater, to 24% to 25% to 30% to 35% to 40% to 50% to 55% to 60% to 70% to 75% to 80% to 90% to 95% to 100% oxygen by gas volume, and/or any combinations of ranges therebetween the aforementioned volume % (inclusive) and/or any fractions thereof (i.e., 21% to 100%, 22% to 100%, 35% to 90%, 90% to 100%, 95% to 100%, etc.). In some specific embodiments, the phospholipid shell may be made up of a main phospholipid, with an acyl chain length ranging from C12 up to C24, and an emulsifier/emulsifier lipid (with non-limiting examples such as PEG-40-sterate and/or DSPE-PEG2000) at ratios ranging from 1:9 up to 9:1. For the purpose of holding and delivering a therapeutic payload for the treatment of wounds, oxygen microbubbles can be loaded with pharmaceuticals either passively through mixing with a pharmaceutical or directly with the functionalization of the oxygen microbubble lipid shell.

In various embodiment, the passive mixing of the oxygen microbubble solution with a solution containing a suspended pharmaceutical or other medicament might produce a useful oxygen microbubble-pharmaceutical solution where the concentration and ratio of oxygen microbubbles to pharmaceutical might not be known a prior. In other embodiments, the direct functionalization of the oxygen microbubble lipid shell for the attachment of a pharmaceutical may allow one to estimate the pharmaceutical payload prior to mixing the oxygen microbubble solution with a pharmaceutical. As one non-limiting example, the oxygen microbubble shell could use a biotinylated emulsifier lipid such as DSPE-PEG2000-BIOTIN in combination with a avidinated pharmaceutical to take advantage of avidin-biotin binding, resulting in the mechanical attachment of pharmaceutical molecules to the surface of the oxygen microbubbles.

In various embodiments, a medicament or other formulant could be chemically bonded in various manners to receptor sites on an inside/outside surface of a microbubble (i.e., the “brush surface” or internal/external chemical bristles of the OMB bubble) as part of the OMB formulation. For example, a microbubble could be outfitted with ligands that bind specific receptors. In many cases, the medicament and/or formulant could include nucleic acids, proteins, gene activated medications, targeted therapeutics, DNA, RNA, etc.

In various embodiments, the disclosed OMB therapies and related devices also offer significant improvements over existing continuous diffusion oxygen (CDO) devices, which use an oxygen concentrator to produce oxygen which is then flowed through a tube to a dressing over the wound site. These CDO devices require significant amounts of fixed power or batteries to operate, CDO devices cannot deliver a combination of other medications with the oxygen and CDO devices require the oxygen concentrator device to be worn or carried by the user. In contrast, the disclosed OMB deliver systems and related therapies can be instituted and maintained with little or no power or additional devices such as an oxygen concentrator. Additionally, various OMB system embodiments can supply and/or provide oxygen directly to a bandage or insert without need for supplemental tubing or inlet and/or outlet ports, if desired. In many embodiments, the structure of the OMB also helps maintain a more moist wound environment in the target tissues when compared to flowing oxygen gas of CDO devices.

In various additional embodiments, an antibiotic, antiseptic, analgesic substance and/or other medicament could be incorporated into the OMB formulation for topical application to the skin wound. A wide variety of antibiotics, treating agents and/or other infection fighting agents are available for topical application, which can include antibiotics suitable for treatment of infections of gram-negative and/or gram-positive bacteria. If desired, a plurality of antibiotic and/or infection fighting agent types can be incorporated, including betadine, peroxide-based preparations, ethacridine lactate, mupirocin (Bactroban), cadexomer iodine, providone iodine, honey-based preparations, silver-based preparations, enzymatic cleansers, chloramphenicol-containing ointments, framycetin sulphate ointment and/or herbal ointments. In various embodiments, a pharmaceutically effective amount of pexiganan cream (commonly known as Locilex 0.8% cream, which is commercially available from Dipexium Pharmaceuticals, Inc., of New York, N.Y.) can be combined with OMBs and topically applied to a surface of the skin ulcer and/or the surrounding healthy tissues. This cream has the ability to kill microbial targets through disruption of the bacterial cell membrane permeability, which is effective against a broad spectrum of gram-positive, gram-negative, aerobic, and anerobic bacteria, as well as fungi, and pexiganan has particular utility against methicillin-resistant Staphylococcus aureus (or MRSA), vancomycin-resistant enterococcus (or VRE), extended-spectrum beta-lactamases (or ESBL) and multi-drug resistant (or MDR) bacteria.

Current approaches toward healing for many types of skin wounds can range from environmental control through dressing applications to surgery in the form of skin grafting and skin flaps. The healing process can depend on the size of the ulcer and patient compliance. Clinically, both deep (full-thickness) and shallow (partial thickness) pressure ulcers and other skin wounds are of concern. In most cases, partial thickness wounds (Grade 1 and 2) can be treated with wound dressings, rather than requiring skin grafts, since the lost epithelium in such “minor” wounds is expected to regenerate on its own with little or no dermal contraction. Immediate concerns with shallow pressure ulcers include blood loss, bacterial invasion, and fluid loss in partial thickness wounds. Shallow wounds typically heal naturally, however, many of these skin ulcers can progress to deeper wounds due to pathology or continual irritation. In any case, speeding up the regenerative healing would be beneficial. Therefore, there is a place for regenerative treatments and/or systems even for these shallow wounds.

Full thickness wounds (Grades 3 and 4) generally involve a loss of the epithelium and dermis. These usually necessitate more active treatments than simple wound dressings. The dermis normally does not naturally regenerate itself, and healing occurs primarily through the development of granulation tissue and scar, causing the wound area to contract and lose its elasticity. In various embodiments, one optimal wound dressing could comprise a dressing that provides a scaffolding structure to promote the development of a new dermis over which the epidermis could grow without any contraction.

A wound dressing, when it is used, can enhance healing in a number of ways. Skin healing can be altered by changing the configuration (pore size, porosity, fiber diameter), the surface (composition, charge, surface energy), the biochemical activity (incorporation of biochemical factors), or the degradation or drug delivery rate of a wound dressing. The goal in virtually all cases is tissue regeneration and at the fastest possible rate. In various embodiments, a wound dressing (if there is one) might desirably be degradable. Dressing change regimens for deep skin ulcers can take from six weeks to six months of bed rest to heal. Typically the choice of last resort, surgical interventions can cause additional unwanted damage to the affected tissues, can result in co-morbidities such as infection or damage at a donor tissue site, and typically involve higher costs for surgery and a lengthy post-operative healing period. Moreover, surgical interventions in the form of pedicle flaps and/or skin grafts may not be ideal solutions. For skin ulcers, skin flaps (the usual method of choice) do not always take and there are a limited number of donor sites available for such tissues.

Various embodiments described herein relate to methods for assessing, and/or treating or ameliorating painful and/or degenerative conditions of the skin, including those that ultimately involve ulcers and/or other skin wounds. Embodiments can include classifications of skin cell and related tissue oxygen/nutrition deficit, pathological conditions and/or associated degeneration and/or chronic conditions that can be based on specific parameters associated with hypoperfusion, hypoxia, and ischemia. Further embodiments can relate to treatments for reducing and/or alleviating the state of hypoperfusion, hypoxia, and ischemia in patients (including local and/or systemic improvements) in which alleviation of said hypoperfusion may lead to therapeutic improvement. Various embodiments described herein can be employed to assess, quantify and/or treat pathologies that can eventually lead to deficient nutrition to and/or waste removal from tissues such as the skin.

Various embodiments described herein can be employed to assess, quantify and/or treat pathologies that can eventually lead to deficient oxygen, nutrition to and/or waste removal from skin layers or other tissues. In one exemplary embodiment, diagnosed dermal hypoperfusion can be treated by increasing the amount of dissolved and/or available oxygen in identified area(s), such as by introduction of an OMB formulation to the surface and/or subsurface of a skin wound and/or on or into healthy tissues proximate to the identified area or areas of injury. If desired, the identified area or areas can be accessed via a transdermal approach with a surgical access and delivery device such as a surgical access needle extending through the patient's skin and overlying soft tissues in a minimally-invasive manner. The OMB formulation could then be introduced into the anatomy through the delivery device.

In another exemplary embodiment, diagnosed dermal injury could be treated by increasing local dissolved oxygen in identified area(s), such as by topical application of a formulation that includes OMB components. In preferred embodiments, the composition could be applied to the surface of the wound or ulcer and/or to the surface of the healthy tissues proximate to the wound or ulcer, as well as to an identified area or areas deficient in oxygen.

In various alternative embodiments, hypoxic and/or ischemic skin disease could be treated by increasing localized dissolved oxygen in the affected area, such as by topical application of a formulation that includes one or more OMBs, by injection of a formulation that includes one or more OMBs below a cutaneous layer, and/or by various combinations thereof. In preferred embodiments, topical application and/or localized injection could be proximate to the wound or ulcer. In other embodiments, introduction of OMB formulations could be undertaken into and/or adjacent to other anatomical structures, including proximate to major arteries and/or veins supplying blood to the affected extremity and/or other skin region, if desired.

In various preferred embodiments, the delivery system could include components of extracellular matrix in combination with the OMB formulation. In some embodiments, said extracellular matrix components may be hyaluronic acid fragments. In other embodiments, said extracellular matrix components may be derivatives of collagen, or perlecan. In various embodiments, the matrix could include a polymer capable of slow release such as a poloxamer block copolymer (Pluronic®, BASF), a basement membrane preparation (Matrigel®, BD Biosciences) or a collagen-based matrix such as described by U.S. Pat. No. 6,346,515, which is incorporated herein by reference.

Wound Healing with Oxygen Microbubble Formulations

Oxygen is one of the basic essentials for sustaining life. Today's medical technology can supply oxygen to patients experiencing pulmonary failure, otherwise known as respiratory failure. Pulmonary failure occurs when the lungs experience significant damage and are unable to supply the body and brain with oxygen. Pulmonary failure may be caused by a variety of conditions including, for example, lung cancer, physical trauma, acute respiratory distress syndrome (ARDS), aerosolized bioterrorism agents, and diseases such as severe acute respiratory syndrome (SARS), pneumonia, tuberculosis, sepsis, and other bacterial or viral infections, physical trauma, and chemical or smoke inhalation. Currently, oxygen can be supplied to patients experiencing pulmonary failure through mechanical ventilation (MV) or extracorporeal membrane oxygenation (ECMO). However, the mortality rate of patients receiving oxygen through MV or ECMO remains high.

Hyperbaric oxygen therapy (HBOT) has been useful in the past in the treatment of problem wounds, including diabetic foot ulcers and late effect radiation injury, wherein a patient is treated with 100% oxygen at higher than atmospheric pressures. In most typical cases, the patient is confined in a specialized chamber for the duration of a treatment (which can last up to three hours or longer), where the patient breathes pure oxygen for an extended period of time to desirably increase oxygen concentrations within the body. While somewhat effective for some wounds, there are a host of potential side effects and complications of HBOT (including the risk of death or serious injury in many cases), and such treatment requires a significant investment in specialized equipment and highly trained personnel.

Another form of therapy proposed to deliver oxygen to damaged tissues is the method of using an oxygen microbubble (OMB) carrier to deliver oxygen directly to tissues and/or the circulatory system. OMBs are oxygen filled bubbles that have a shell composed of a phospholipid monolayer. The phospholipid monolayer shell of an OMB has similar composition to lung surfactant and requires comparable physical properties, such as rapid adsorption to and mechanical stabilization of the gas/liquid interface and high gas permeability. Thus OMBs are also designed to mimic the mechanical and gas transport properties of the alveolus to deliver the oxygen payload and uptake carbon dioxide.

In addition to increasing the local concentration of oxygen proximate to a wound bead, it is believed that OMB's can facilitate various levels of oxygen and/or carbon dioxide exchange across the tissues in and/or proximate to a targeted wound bed (potentially including across cellular boundaries of a wide variety of body tissues). Moreover, the introduction of OMBs to a surface and/or subsurface of a wound bed is not subject to a strict upper limit of the microbubble size and volume fraction, since the microbubbles are not intended to be directly injected into a vascular channel (in many cases) and thus can safely ripen, burst or otherwise degrade and/or be removed from a target anatomy as desired. These methods desirably provide oxygen to a subject to facilitate healing of the wound.

OMB Production

In an exemplary embodiment, lipids can be mixed at a 9:1 molar ratio of distearoyl phosphatidylcholine (DSPC) to poly(ethylene glycol)-40 stearate (PEG40S) in saline and sonicated at low power to create the small, unilamellar liposomes. O2 and liposomes (5 mg/mL) are then combined in the reaction chamber, where a high-power, ½-inch diameter, 20-kHz sonicator tip emulsifies the oxygen gas into micrometer-scale spheres around which phospholipid adsorbs from vesicles and micelles and self-assembles into a highly condensed (solid) monolayer coating. OMBs are separated from macroscopic foam in a subsequent flotation container and collected in syringes and centrifuged (500 g for 3 min) to form concentrated OMBs. The sonication chamber and container are jacketed with circulating coolant to maintain a constant temperature of 20° C.

OMBs can be fabricated for live animal testing and/or therapeutic use in humans and other mammals. In some embodiment, four factors can be investigated: perfusate, perfusion rate, motility drug, and method (IP or GI). OMB perfusate at 70% and 90% indicate the volume fraction of oxygen in the OMB emulsion. At 70%, the OMB emulsion's rheological properties are similar to saline and the perfusate is expected to circulate well through common delivery techniques to the body including methods associated with peritoneal dialysis, or in delivery to cavities of the body, or delivery to tissue via a natural orifice and/or access point into the body (i.e., an OMB enema or other treatment without incision—such as via rectal enema, colonic irrigation device, endoscopes, gastroscopes, bronchoscopes, laryngoscopes, cytoscopes, duodenoscopes, ureteroscopes, hysteroscopes, stomach feeding tube, access to, stomach, intestine).

The OMB size distribution can be varied in a variety of ways, including by choosing different residence times in the flotation container (e.g., 153 min for a 10-μm diameter cut-off; 38 min for a 20-μm diameter cut-off). Size distribution is measured, for example, by electrical capacitance, light extinction/scattering, flow cytometry scatter, and optical microscopy. Alternatively, size selection may be unnecessary and may be removed from the process. OMB volume fraction is measured, for example, by gravimetric analysis and varied from 50-90 vol % by dilution with saline. Microbubble size and concentration is measured over time to investigate coalescence, Ostwald ripening and stability in storage.

OMB Bandages and Related Systems

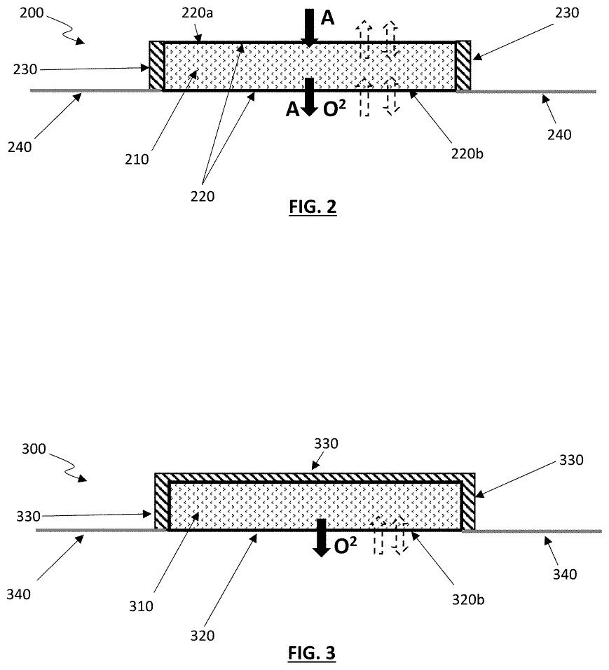

FIG. 2 depicts one exemplary embodiment of a dressing or bandage 200 that incorporates an OMB compound for application to a skin wound. The bandage 200 can desirably include a reservoir 210, which in this embodiment contains an oxygen microbubble (OMB) formulation. The reservoir can be bounded on at least one side by a gas and/or liquid-permeable membrane 220 such as Gor-Tex™ (commercially available from W.L. Gore and Associates of Elkton, Md., USA), Tyvek (commercially available from E.I. de Pont de Nemours and Company of Wilmington, Deleware, USA) or other materials well known in the art. The remaining sides of the reservoir can be formed from impermeable or gas-tight membranes 230, such as a variety of plastics, polymers and/or metal foils, as known in the art. The bandage 200 can also include an adhesive fringe or border 240, which desirably allows the bandage to be connected to healthy tissues adjacent to the wound in a known manner.

In the embodiment shown in FIG. 2, the reservoir 210 is bounded on an upper surface by a first gas-permeable membrane 220a, and on a lower surface by a second gas-permeable membrane 220b (which may be formed from the same or a different material than the first gas permeable membrane), which can desirably allow atmospheric air “A” to penetrate into and/or pass through the reservoir 210 to contact the wound surface (which could include direct OMB contact with the wound surface and/or wound contact with the free O2 gas itself—not shown). Desirably, oxygen “O” or “O²” from the OMB formulation in the reservoir 210 will also be able to pass through at the least the second gas-permeable membrane 220b, thereby increasing the local oxygen concentration proximate to the wound bed in a desired manner. In various alternative embodiments, the flow of gases through the one or more gas-permeable membranes 220a and/or 220b could alternatively be unidirectional and/or bidirectional (or any combination thereof—depicted as dotted-line flow arrows in FIGS. 2, 3 and 4), which could allow for gas flow from the wound bed into the reservoir (i.e., carbon-dioxide transport) back through gas-permeable membrane 220b and/or flow of gases out of the reservoir into the surrounding atmosphere (i.e., oxygen and/or carbon-dioxide flow out of the bandage reservoir) back through gas-permeable membrane 220a.

FIG. 3 depicts an alternative embodiment of a dressing or bandage 300 that incorporates an OMB compound for application to a skin wound. The bandage 300 can desirably include a reservoir 310, which in this embodiment contains an oxygen microbubble (OMB) formulation. The reservoir can be bounded on one side by a gas-permeable membrane 220 such as Gor-Tex™ (commercially available from W.L. Gore and Associates of Elkton, Md., USA), Tyvek (commercially available from E.I. de Pont de Nemours and Company of Wilmington, Deleware, USA) or other materials well known in the art. The remaining sides of the reservoir can be formed from impermeable or gas-tight membranes 330, such as a variety of plastics, polymers and/or metal foils, as known in the art. The bandage 300 can also include an adhesive fringe or border 340, which desirably allows the bandage to be connected to healthy tissues adjacent to the wound in a known manner.

In the embodiment shown in FIG. 3, the reservoir 310 is bounded on three sides by the impermeable or gas-tight membrane 330, with only a lower side having a gas and/or vapor/liquid-permeable membrane 320, which can desirably allow Oxygen (“O²”) from the OMB formulation to contact the wound surface (which could include direct OMB contact with the wound surface and/or wound contact with the free O2 gas itself—not shown). Desirably, oxygen “O” or “O2” and/or individual microbubbles from the OMB formulation in the reservoir 310 will be able to pass through the gas-permeable membrane 320, thereby increasing the local oxygen concentration proximate to the wound bed in a desired manner and/or transporting oxygen directly to (and potentially accepting carbon-dioxide from) the walls of cells in the wound bed.

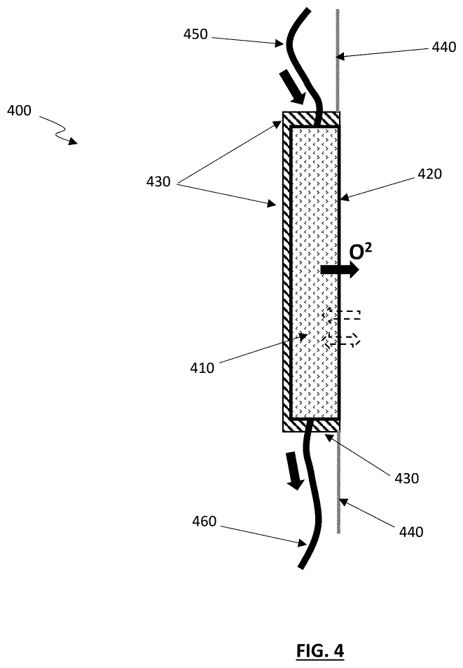

FIG. 4 depicts another alternative embodiment of a dressing or bandage 400 that incorporates an OMB compound and related flow system components for application to a skin wound. The bandage 400 can desirably include a reservoir 410, which in this embodiment contains an oxygen microbubble (OMB) formulation. The reservoir can be bounded on one side by a gas-permeable membrane 420 such as Gor-Tex™ (commercially available from W.L. Gore and Associates of Elkton, Md., USA), Tyvek (commercially available from E.I. de Pont de Nemours and Company of Wilmington, Deleware, USA) or other materials well known in the art. The remaining sides of the reservoir can be formed from impermeable or gas-tight membranes 430, such as a variety of plastics, polymers and/or metal foils, as known in the art. The bandage 400 can also include an adhesive fringe or border 440, which desirably allows the bandage to be connected to healthy tissues adjacent to the wound in a known manner.

In at least one alternative embodiment, the dressing or bandage of FIG. 4 could be formed without a gas-permeable membrane 420, such as where the OMB compound and/or any related components may be relative viscous, “self-supporting” and/or capable of adhering and/or being adhered to the bandage itself and/or any common gauze cotton or line materials located thereon. In such a case, it may not be necessary or desirous to have a discrete barrier material located between the reservoir and the treated tissue surface. In some embodiment, the OMB formulation may simply be placed on the treated tissue with a bandage optionally placed thereover, or the OMB formulation may be placed on the bandage itself before the bandage is placed over the tissue to be treated. In some embodiments, the OMB formulation may comprise a thixotropic material. In some embodiments, the OMB formulation may be positioned within openings or pores of a permeable material such as a knitted, woven and/or felted reservoir that contains some portion and/or all of the OMB formulation.

In the embodiment shown in FIG. 4, the reservoir 410 is bounded on three sides by the impermeable or gas-tight membrane 430, with only a lateral side having a gas and/or vapor/liquid-permeable membrane 420, which can desirably allow Oxygen (“O²”) from the OMB formulation to contact the wound surface (which could include direct OMB contact with the wound surface and/or wound contact with the free O2 gas itself—not shown). Desirably, oxygen “O” or “O2” from the OMB formulation in the reservoir 410 will be able to pass through the gas-permeable membrane 420, thereby increasing the local oxygen concentration proximate to the wound bed in a desired manner.

The device of FIG. 4 can further include one or more OMB infusion inlet(s) 450 and/or one or more OMB/saline infusion outlet(s) 460, which desirably facilitates the introduction of “fresh” oxygen microbubbles via the inlet 450 and/or the removal of “used” microbubbles and/or liquid saline via the outlet 460 (which may contain waste products and/or waste gasses such as carbon dioxide from the wound bed and/or unutilized oxygen). In various embodiments, this arrangement could potentially induce a “flow” of OMBs in the reservoir and/or across the wound bed. In various embodiments, this will allow gas diffusion from high concentration to low concentration into the wound as a means of O2 delivery to tissues and can also allow potentially direct transmembrane transfer of oxygen through the OMB membranes and/or adjacent tissue cell membranes. In addition, the disclosed arrangement could allow the use of different types and or formulas of OMBs to be utilized in a single bandage, including the periodic modification of the OMB formulation to accommodate changing conditions within the wound bed.

In various embodiments, a periodic and/or continuous flow of OMBs will allow a potential for some of the OMBs to degenerate and/or “pop” in situ, thus releasing their oxygen payload and/or saline structure to the tissue surface, which may be advantageous for various uses (including sonoporation and/or similar energy-related treatments, for example).

If desired, the system of FIG. 4 could provide OMBs at atmospheric pressures, or at one or more pressures elevated above or depressed below atmospheric pressure (i.e., pressurized or vacuum delivery), or various combinations thereof, including methods involving intermittent changes between two or more pressures and/or vacuums. In some cases, the periodic application of a vacuum to the OMBs might cause bursting or other degradation of the OMBs, thereby releasing their oxygen and/or saline cargoes at a desired location and/or time during the wound therapy, with subsequent additional OMBs then introduced through the inlet at atmospheric pressure to fill the reservoir, followed by another vacuum period and cargo release, etc.

If desired, the infusion and/or drain system of FIG. 4 could comprise a fixed and/or mobile powered system (i.e., using a metering pump), or could comprise a fixed and/or mobile unpowered system that utilizes a gravity feed reservoir bag to provide OMB flow through the inlet(s) in a manner similar to a mobile saline drip.

Desirably, in the various exemplary embodiments described herein, the OMB formulation can comprise 95% to 100% O₂ by gas volume and/or 70% to 90% O² by total volume of the compound, although a wide variety of gas volume and/or total volume percentages could potentially be useful, depending upon application and/or wound type and/or environment. In various alternative embodiments, the oxygen microbubble formulation described herein can be tailored to comprise from 21% oxygen (the percentage of oxygen typically present in atmospheric air) up to 100% oxygen by gas volume, which can result in a formulation that consists of 1% to 75% oxygen by total volume. This resulting oxygen microbubble formulation can consist of 99% to 25% aqueous lipid solution. The ratio of oxygen to lipid solution ranging from 1:99 to 75:25 is dependent on the concentration of oxygen microbubbles within the suspension as well as the concentration of oxygen within the gas used during the formation of the oxygen microbubbles. Lower amounts of oxygen microbubbles result in lower amounts of oxygen and higher amounts of lipid solution. Higher amounts of oxygen microbubbles result in greater amounts of oxygen and lower amounts of lipid solution. Oxygen microbubbles can be concentrated in suspension passively through floatation or forcefully through centrifugation. Additionally, oxygen microbubbles can be used as an additive to topical lotions for the purpose of wound treatment. When combined with an ointment at ratios of 1:9 up to 9:1, oxygen microbubble ointments can contain oxygen by gas volumes ranging from 21% to 100%. These resulting oxygen microbubbles ointments would desirably contain oxygen percent by total volumes ranging from 0.1% to 67.5%.

In various embodiments, an OMB compound can be included as part of a treatment regime for a skin wound, ulcer or other chronic skin condition. Such treatment can include topical application of the OMB compound to the surface of the wound, to the margin(s) of the wound and/or to the surface of surrounding healthy and/or undamaged skin or other tissues. Desirably, the OMB compound will induce cell growth, wound healing and/or growth and/or expansion of the various vascular structures/network underlying and/or adjacent to the damaged tissues, providing improved oxygen to the damaged and/or repairing cells and potentially improving the condition of the underlying vascular network to improve the longer term supply of oxygen, nutrients and/or waste removal for at least a portion of the damaged tissues.

In various embodiments, an OMB compound applied to a wound or other skin surface may have the potential to improve the level(s) of oxygen in body tissues more distal from the application site, such as where diffusion and/or vascular circulation of oxygen from the OMB compound occurs. In still other embodiments, the OMB compound might induce the vasculature to repair, bypass and/or reroute a damaged and/or degraded area of the anatomy, thereby potentially improving localized and/or systemic vascular flow within the extremity and/or other anatomical area of the patient's body. The presence of an elevated level of oxygen at the surface of tumors of the skin may reduce tumor hypoxia and decrease vascular growth within the tumor, slowing the rate of tumor growth and allowing greater treatment windows for tumor therapy.

In addition to the various effects described herein, in various embodiments the application of an OMB compound to a damaged skin structure will desirably induce growth and/or repair of cells of the various skin layers, including within one or more of the dermal fibroblasts, the vascular endothelial cells and/or the epidermal keratinocytes. For example, application of an OMB compound may markedly increase the proliferation of fibroblasts that give rise to granulation tissue, which fills up a wound space/cavity early in the wound healing process. Moreover, the OMB compound may activate and/or signal a cascade of cell proliferation, such as by initiating the biological signals of FGF2 and FGF7, which in turn signal additional healing responses.

### Treatment

As used herein, the terms “treating,” “treatment,” “therapeutic,” or “therapy” do not necessarily mean total cure or abolition of the disease or condition. Any alleviation of any undesired signs or symptoms of a disease or condition, to any extent, can be considered treatment and/or therapy. It is entirely possible that “treatment” consists of a temporary improvement of the damaged tissues, with additional repeated treatments required over time to continue the regenerative process and/or to prevent further degradation.

In various embodiments, skin wounds, ulcers and/or other conditions can be treated by application and/or administration of a medical device that generates a periodic or continuous release of OMB compounds onto tissue, into tissue and/or into blood and/or fluid circulation so as to reduce tissue degradation and/or promote a healing response, and specifically, collateralization in area(s) proximal to the skin condition. In some embodiments, the composition might further include stem cells and/or other biological treatments, which might be used in conjunction with OMB's prior to, during and/or subsequent to the employment of tissue grafts to repair or replace native tissues. If desired, such compositions could be used to prepare a patient's anatomical site for an intended tissue graft or surgical procedure, could be used to prepare the tissue graft for implantation, and/or could be used to treat the patient and/or tissue graft site after implantation.

In various embodiments, a medical device may include a reservoir, a slow release pump and/or some other supply device, which could include external devices as well as implantable indwelling or osmotic pumps or localized delivery systems. In various embodiments, the device may incorporate a polymer capable of slow release of materials incorporated therein.

In various embodiments, the composition delivered by the medical device contains not only a therapeutically sufficient concentration of OMBs, but also a chemotactic agent. The chemotactic agent recruits cells capable of causing or promoting angiogenesis. In some embodiments, a chemotactic agent such as stromal cell-derived factor 1 (SDF-1) could be included in the composition with the OMB. In various embodiments, the composition delivered by the medical device may contain an anti-inflammatory agent at a concentration sufficient for inhibiting possible inflammatory reactions associated with wound healing, while at the same time not inhibiting collateral blood vessel formation. If desired, the various agents described herein could be combined with various scaffolds and scaffolding structures, as well as stem cells, which can include embryonic stem cells and/or adult stem cells, as desired.

In various embodiments, the treatment of patients could include various combinations of active and passive treatment phases, wherein active treatment phases desirably induced a positive effect on healing of the patient's ulcer, which might even include improved healing effects in one of both of the active and/or passive phases.

In another example, the topical application of an OMB compound to a wound or skin ulcer of a patient suffering from chronic diabetic ulcers can significantly increase the rate of healing of the wound/ulcer during the active treatment phase (as compare to a placebo or non-treatment group), but can potentially also induce significantly improved skin healing effects during a follow-on “non-treatment” phase (i.e., passive treatment phase) after cessation of the active treatment. One exemplary treatment regime for a series of patients suffering from wound and/or diabetic chronic ulcers could comprise topical application of an OMB compound to the patients' skin ulcers at a frequency of three times a week, for a period of three weeks.

In another exemplary embodiment, an OMB compound could be incorporated into and/or applied to a fibrin matrix and could be applied topically to a skin wound and/or surrounding external tissues, which desirably significantly accelerates the healing process of the skin wound and leads to significant improvement in healing, with complete epithelialization and minimal contraction, as compared to a natural, healthy healing response.

In various alternative embodiments, an OMB compound might be injected and/or otherwise introduced beneath the external surface of the wound, such by injection via a hypodermic needle into a subsurface structure of the center of the wound, the wound margin and/or into underlying and/or adjacent healthy tissues. If desired, concurrent and/or alternating surface and subsurface treatments (including as previously described) could be undertaken. In a similar manner, treatment of internal body tissues using an OMB formulation might be accomplished by injection through the skin and/or other tissues and/or may be effectuated via natural pathways into the body such as through the gastrointestinal tract. For example, stomach ulcers and/or other tissue pathologies might be directly treated via the alimentary canal, such as with a medical device extending down the patient's esophagus and into the stomach, duodenum and/or upper intestine. As another alternative, access to the large intestine and/or lower small intestine may be accomplished via a rectal approach, using a proctoscope and/or enema nozzle. In some embodiments, such as for treatment of ulcers in the digestive tract, a continuous flow of OMB through the digestive system may increase oxygen presence on the surface of an internal ulcer and promote healing response at the internal tissue disruption. In a similar manner, injuries to the walls of the large intestine might be treated using a continuous flow of OMB through the colon may increase oxygen presence on the injury and promote healing response. In various embodiments, these treatments may also have an added benefit of transferring additional oxygen to and/or removing carbon dioxide from the patient's bloodstream, especially where the OMB formulation contacts the walls of the stomach, the small intestine and/or the large intestine. If desired, various treatments may utilize an enteroscope or similar device to advance the device to a desired location within the digestive tract, and in some embodiments one or more balloons or other devices could be inflated within the tract to desirably artificially seal a portion of the passage wherein the OMB compound could be introduced and/or circulated for a desired treatment and/or period of time.

In various treatments, the size, shape and/or condition of the skin ulcer might predispose the wound to a particular treatment or combination or treatments. For example, for a skin ulcer presenting less than an approximately 6 cm² external surface, a topical compound might be more appropriate for treatment. However, where the ulcer may be greater than approximately 6 cm², or where the skin ulcer includes damage to underlying bone, tendon or cartilage, it may be desirous to combine a surface treatment of the ulcer with one or more injections of an OMB compound into the ulcer, into the tissue region proximate to the margin between the ulcer and surrounding healthier tissues, and/or into healthier tissues surrounding the ulcer.

In a similar fashion, chronic wounds or ulcers, such as diabetic foot ulcers, or other wounds known to be of ischemic origin, could be treated in various combination approaches. For example, if cells, scaffolds, signaling proteins such as various growth factors, genes or any other tissue or synthetic transplantation were contemplated to be utilized in an area of ulcer on the diabetic foot or other area of anatomy that is suffering with a chronic ischemic wound, then proper pre-treatment using an OMB compound might be utilized prior to the introduction of such materials.

Topical Application, Wound Moistening and Reduced Absorption

In various embodiments, an added benefit of topical therapy of an OMB compound as a primary treatment modality is the presence of saline in various embodiments of the OMB formulation as a carrier, wherein degradation and/or “popping” of the microbubbles can release the saline and/or any other materials forming the bubble walls to the surface of the wound bed. This not only moisturizes and/or lubricates the wound surface and/or sub-surface but can also facilitate absorption of saline and/or medicaments into the wound bed. Surface application of the OMB formulation can also provide a reduced opportunity for absorption of the of the complimentary medicaments in the OMB formulation into the patient's blood stream, where systemic distribution of such medicaments outside of the wound and/or damaged skin region may be undesirable.

Bandage or Dressing for Damaged Tissue and/or Wounds

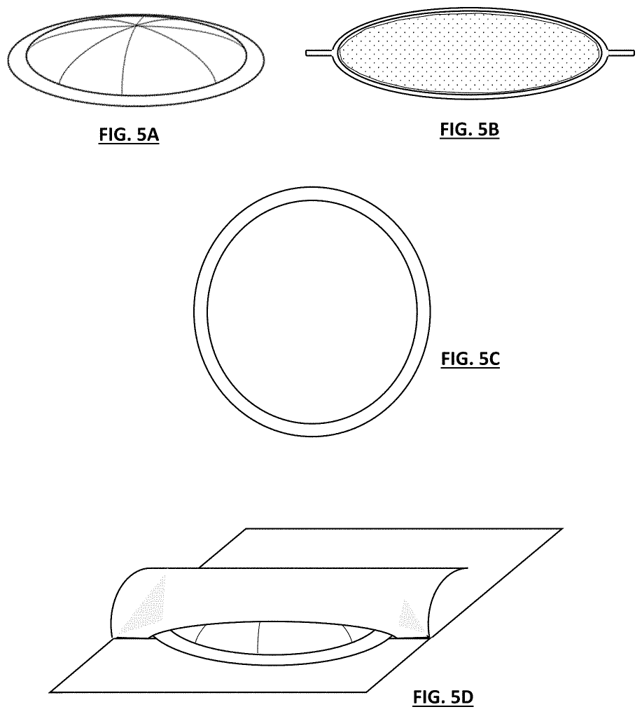

FIGS. 5A through 5C depicts various views of one embodiment of an insert or pad that can serve as a “reservoir” of an OMB formulation and/or compound and/or other medicaments for topical treatment of the skin ulcer. In this embodiment, the insert can include a central portion for containing the various medicaments, with at least one outer skin-facing surface comprising a membrane that is permeable to the various medicaments. Desirably, a medicament contained within the central portion can pass through the permeable membrane and onto the surface of the insert, which can then transfer the OMBs and/or the medicament to the surface of the skin ulcer and/or surrounding tissue via direct contact. In various embodiments, a flexible porous, spongy or other medicament retaining material can be positioned within the central portion, with the various medicaments contained within and/or incorporated into the material. If desired, an adhesive, hook and loop fasteners, or other retaining arrangement could be provided on the insert to retain the insert in a desired location and/or position on the targeted anatomy.

In various embodiments, the insert might desirably experience pressure or stress during the patient's activities of normal daily living (i.e., walking, weight bearing on a limb or an appendage, or localized pressure from bed rest or sitting posture), with various patient actions resulting in compressing, squeezing or otherwise impelling the OMBs and/or the medicament retaining material to expel some portion of the OMBs and/or the medicament through the membrane and into contact with the wound or surrounding skin surface. Desirably, such expelling action can occur on an occasional and/or continuous basis during daily activities, with the added benefit of reapplication of OMB medicament to the ulcer and surrounding tissues on a periodic basis without requiring direct patient interaction. If desired, inserts could be individually packaged, such as by being enclosed in a “peel pouch” or similar packaging (see FIG. 5D). Alternatively, the inserts could be bulk packaged in a resealable container, if desired.

In various embodiments, the entire outer covering of the insert (and/or the entirety of the insert) might comprise a flexible material, with one or more outer surfaces of the insert comprising a medicament permeable layer. Alternatively, portions of the insert material could comprise non-permeable flexible materials, such as some or all of the “back” surface of the insert, wherein the front surface may be intended to be in contact with the patient's skin. If desired, this back surface could alternatively comprise a hard, relatively inflexible material, if desired.

In various embodiments, an insert could be used individually by the patient or caregiver to apply medicament directly to a skin wound or ulcer. In such a case, the insert could be used in a manner similar to antiseptic wipes, with a single insert used to treat multiple wounds, if desired, and then discarded after such use. In another alternative embodiment, the insert could be used to topically apply medicament to one or more skin wounds (i.e., wiped over the skin wounds), and then the insert could be placed over one wound to provide longer-term treatment for that wound and/or for treatment of adjacent wounds/ulcers, if desired. In this manner a single insert could be useful in treating multiple wounds/ulcers and/or could be used to treat skin areas in danger of developing ulcers, along with the primary wound/ulcer treatment using the insert on a “longer term’ basis, as described previously.

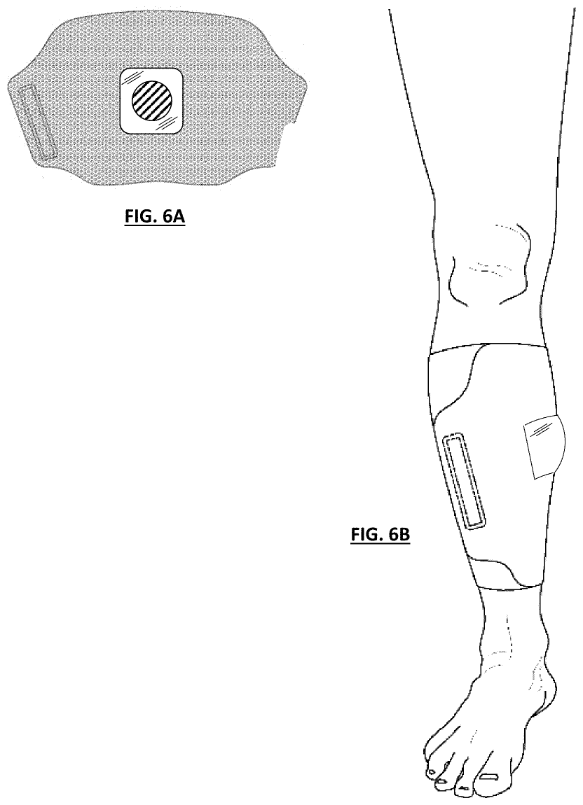

FIG. 6A depicts an alternative embodiment of a prosthesis for use in treating skin ulcers and other wounds with OMB compounds. This embodiment comprises a securement or compression-type bandage or wrap, with a pouch or pocket for accommodating an OMB/medicament insert. Desirably, the pouch will include a clear or transparent portion, such as a central portion of the pouch and/or a pouch periphery region, which desirably allows the patient or a caregiver to view some portion of the skin surface underlying the pouch. In addition, the inner surface of the pouch will desirably allow medicament from the insert to pass through and/or around the pouch material and contact the underlying skin wound and/or adjacent tissue. Desirably, once an insert is placed into the pouch, the prosthesis can be positioned over the skin wound requiring treatment, with the clear portion allowing visual verification that the insert is properly positioned over the wound. Alternatively, the prosthesis could first be positioned over the skin wound requiring treatment, with the clear portion allowing visual verification that the skin wound is in a desired position relative to the pouch region, and then the insert can be placed into the pouch. Various embodiments for use with extremities could include a leg prosthesis (FIG. 6B) and/or other designs.

In various embodiments, patient movement and/or patient actions can desirably result in compressing, squeezing or otherwise impelling the medicament retaining material within the insert to expel some portion of the OMBs and/or medicament through the permeable insert membrane and into contact with the wound or surrounding skin surface. Alternatively, the patient could apply external direct pressure to the pouch for a short time on a periodic basis (i.e., by pressing their opposing hand down on the outer surface of the pouch), which would desirably re-apply and/or refresh the OMB/medicament to the surface of the skin wound.

In various alternative embodiments, a prosthesis for use in treating skin could comprise an adhesive bandage or pad, with a pouch or pocket formed therein. The pad could be adhered to the skin of the patient, if desired, with an insert contained within the pouch at a location adjacent to the skin wound or ulcer. As previously described, the patient's movement and/or outside forces could impel the insert to extrude, exude and/or otherwise deliver the OMBs/medicament to the surface of the skin wound and optionally to adjacent healthy skin tissue. Desirably, some portion of the pouch will be transparent, allowing the patient or a caregiver to view the skin wound or ulcer through the transparent portion to facilitate wound assessment and/or proper positioning of the insert.

In various embodiments the pouch could include a closeable opening on either or both of the back side and/or skin facing side of the prosthesis. The ability to remove and replace the insert without removing the prosthesis from the treated anatomy may be particularly useful in certain situations, such as where removal and replacement of the prosthesis would be difficult for the patient to accomplish unassisted (i.e., where the prosthesis is on an arm, or in a location not directly reachable and/or viewable by the patient). In such cases, the removal and replacement of the insert from the exterior of the prosthesis can allow the patient to easily self-administer a new dose of the OMB formulation in an outpatient setting.

Burns and/or Other Skin Wounds

Various embodiments described herein could also have particular utility with regards to various types of damaged and/or injured surface and/or subsurface skin tissues, including surface/subsurface skin tissue burns due to excessive heat, excessive cold, chemical contact, radiation effects, wind abrasion and/or otherwise induced tissue damage. It should be understood that the various assessment and/or treatment modalities described herein could be utilized in conjunction with the treatment and/or management of such wounds, including various combinations of the various embodiments disclosed herein.

Other Joints, Organs and Tissues

The various embodiments described herein, including the treatments thereof using various tools, techniques and surgical methods can be applied to various other tissues in a human or animal body, including any soft or hard tissues including, without limitation, joint tissues, a spine, an elbow, a shoulder, a wrist, a hand, a finger, a jaw, a hip, a knee, an ankle, a foot, or a toe joint. In a similar manner, various alternative embodiments and/or modifications thereof could be used for the treatment of soft tissue structures and/or other organs, including organs/structures within the body such as the heart, heart tissue grafts and/or heart transplants, and/or structures within the body and/or digestive tracts such as internal stomach surfaces (i.e., stomach ulcers), intestinal lesions, hemorrhoids, and/or ulcers of the small and large intestines, etc.

### Headings

The headings provided herein are merely for the reader's convenience and should not be construed as limiting the scope of the various disclosures or sections thereunder, nor should they preclude the application of such disclosures to various other embodiments or sections described herein.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention. The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the claims provided herein.

What is claimed is:

1. A method for supplying oxygen to a damaged integumentary tissue, comprising:
topically applying an effective amount of a compound comprising a suspension comprising microbubbles and a carrier proximate to a surface of the damaged integumentary tissue, wherein the microbubbles comprise a lipid envelope and a gas core, wherein the gas core comprises oxygen, and wherein the suspension comprises at least 40% oxygen by volume,
wherein the effective amount of the topical compound increases the concentration of oxygen in the damaged integumentary tissue.

2. The method of claim 1, wherein the damaged integumentary tissue comprises a surface skin tissue of a patient.

3. The method of claim 1, wherein the damaged integumentary tissue comprises a burned skin surface of a patient.

4. The method of claim 1, wherein the damaged integumentary tissue comprises a diabetic foot ulcer.

5. The method of claim 1, wherein the damaged integumentary tissue comprises a tissue site within the gastrointestinal tract of a patient.

6. The method of claim 1, wherein the step of topically applying the compound proximate to the surface of the damaged integumentary tissue comprises placing a wound dressing impregnated with the microbubble compound into intimate contact with the surface of the damaged integumentary tissue.

7. The method of claim 1, wherein the step of topically applying the compound proximate to the surface of the damaged integumentary tissue comprises placing a tissue graft material into intimate contact with the damaged integumentary tissue and applying the compound directly to at least a portion of a surface of the tissue graft material.

8. The method of claim 1, wherein the step of topically applying the compound proximate to the surface of the damaged integumentary tissue comprises inducing a continuous flow of the microbubble compound across the damaged integumentary tissue.

9. The method of claim 1, wherein the compound further comprises a medicament for treatment of the damaged integumentary tissue.

10. The method of claim 1, wherein the medicament comprises an anti-inflammatory compound.

11. A method for supplying oxygen to an integumentary tissue, comprising:
applying an effective amount of a compound comprising a suspension comprising microbubbles and a carrier to a surface of the integumentary tissue, wherein the microbubbles comprise a lipid envelope and a gas core, wherein the gas core comprises oxygen, and wherein the suspension comprises at least 40% oxygen by volume,
wherein the effective amount of the topical compound increases the concentration of oxygen in the integumentary tissue.

12. The method of claim 11, wherein the integumentary tissue comprises a surface skin tissue of a patient.

13. The method of claim 11, wherein the effective amount of the topical compound increases a blood oxygen level in a vascular system proximate to the integumentary tissue.

14. The method of claim 13, wherein the integumentary tissue comprises a tissue site within the gastrointestinal tract of a patient.

15. The method of claim 14, wherein the step of applying the compound to the surface of the tissue site within the gastrointestinal tract of the patient comprises inducing a continuous flow of the compound across the surface of the tissue site.

16. The method of claim 15, wherein a colonic irrigation device is utilized to induce the continuous flow of the compound across the tissue site.

17. A method for increasing the oxygen level within tissues proximate to a damaged tissue site, comprising:
topically applying a microbubble compound to the tissues proximate to the damaged tissue site, the microbubble compound comprising a suspension comprising microbubbles and a carrier, wherein the microbubbles comprise a lipid envelope and a gas core, wherein the gas core comprises oxygen, and wherein the suspension comprises at least 40% oxygen by volume,
wherein the microbubble compound increases the concentration of oxygen in the tissues proximate to the damaged skin tissue site.

18. The method of claim 17, wherein the tissues proximate to the damaged tissue site comprise healthy skin tissues.

19. The method of claim 17, wherein the tissues proximate to the damaged tissue site comprise tissues within the damaged tissue site.

20. The method of claim 19, wherein the topical application of the microbubble compound to the tissues within the damaged tissue site further hydrates the damaged tissue site.
